(19) United States
(12) Patent Application Publication
(10) Pub. No.: US 2014/0142682 A1
(43) Pub. Date: May 22, 2014

(54) IMPLANTABLE BIOCOMPATIBLE TUBULAR MATERIAL

(71) Applicant: W. L. Gore & Associates, Inc, Newark, DE (US)

(72) Inventor: Rachel Radspinner, Flagstaff, AZ (US)

(73) Assignee: W.L. Gore & Associates, Inc, Newark, DE (US)

(21) Appl. No.: 13/963,733

(22) Filed: Aug. 9, 2013

Related U.S. Application Data
(60) Provisional application No. 61/682,070, filed on Aug. 10, 2012.

(51) Int. Cl. A61F 2/82

(52) U.S. Cl. CPC A61F 2/82
USPC 623/1.15

(57) ABSTRACT

The present disclosure describes medical devices comprising a biocompatible tubular material. Such devices can include graft members for implanting in the vasculature of a patient. The tubular material of these graft members can be relatively thin, while providing comparable or improved performance over conventional graft members.

### CROSS-REFERENCE TO RELATED APPLICATIONS

This application claims priority to U.S. Provisional Application No. 61/682,070, entitled “IMPLANTABLE BIOCOMPATIBLE TUBULAR MATERIAL” filed on Aug. 10, 2012, which is hereby incorporated by reference in its entirety.

### FIELD

The present disclosure relates generally to implantable, biocompatible materials and, more specifically, to medical devices comprising thin, flexible, durable, and biocompatible tubular materials.

### BACKGROUND

Implantable medical devices are frequently used to treat the anatomy of patients. Such devices can be permanently or semi-permanently implanted in the anatomy to provide treatment to the patient. Frequently, these devices, including stents, grafts, stent-grafts, filters, valves, occluders, markers, mapping devices, therapeutic agent delivery devices, prostheses, pumps, bandages, and other endoluminal and implantable devices, are inserted into the body at an insertion point and deployed to a treatment area using a catheter.

However, the insertion point of the medical device can become infected or irritated, with a higher risk of complications often corresponding to the greater the size of the crossing profile. The crossing profile is generally determined by the cross sectional area of the medical device in its delivery state. Thus, reducing the size of the medical device and hence, the crossing profile, can improve healing and potentially reduce the possibility of infection. Additionally, by reducing the crossing profile, additional benefits such as increased flexibility and steerability, increased transparency, increased tear resistance, reduced frictional forces, reduced surface area, and increased crushability, among others, may be achieved.

However, reducing the size of the medical device by, for example, reducing the thickness of a graft member used in connection with the medical device, typically results in a reduction or trade-off of desirable properties of the graft member. For example, among other properties, burst strength, maximum load, and abrasion resistance may be compromised.

Accordingly, there is a need for medical devices that feature a thinner graft member that performs as well or better than conventional graft members.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description, serve to explain the principles of the disclosure, wherein;

FIG. 1 illustrates a perspective view of a medical device in accordance with the present disclosure;

FIGS. 2A-2D illustrate perspective views of medical devices in accordance with the present disclosure;

FIGS. 3A and 3B illustrate perspective views of medical devices in accordance with the present disclosure;

FIGS. 4A-4C illustrate perspective views of a medical device in accordance with the present disclosure;

FIGS. 5A-5F illustrate SEM images of membrane materials in accordance with the present disclosure;

FIG. 6 is a graph comparing attributes of medical devices in accordance with the present disclosure;

FIG. 7 is a graph comparing attributes of medical devices in accordance with the present disclosure;

FIG. 8 is a graph comparing attributes of medical devices in accordance with the present disclosure;

FIG. 9 is a graph comparing attributes of medical devices in accordance with the present disclosure;

FIG. 10 is a graph comparing attributes of medical devices in accordance with the present disclosure;

FIG. 11 is a graph comparing attributes of medical devices in accordance with the present disclosure;

FIG. 12 is an illustration of the relative cross sectional areas of a prior art medical device and a medical device accordance with the present disclosure;

FIG. 13 is a graph illustrating the relationship between graft member thickness and the area of the delivery profile of medical devices in accordance with the present disclosure; and

FIG. 14 is a chart summarizing the various attributes of medical devices in accordance with the present disclosure.

### DETAILED DESCRIPTION OF THE ILLUSTRATED EMBODIMENTS

Persons skilled in the art will readily appreciate that various aspects of the present disclosure can be realized by any number of methods and systems configured to perform the intended functions. Stated differently, other methods and systems can be incorporated herein to perform the intended functions. It should also be noted that the accompanying drawing figures referred to herein are not all drawn to scale, but can be exaggerated to illustrate various aspects of the present disclosure, and in that regard, the drawing figures should not be construed as limiting.

As used herein, “medical devices” can include, for example, stents, grafts, stent-grafts, filters, valves, occluders, markers, mapping devices, therapeutic agent delivery devices, prostheses, pumps, bandages, and other endoluminal and implantable devices that are implanted, acutely or chronically, in the vasculature or other body lumen or cavity at a treatment region. Such medical devices can comprise a flexible material that can provide a fluid-resistant or fluid-proof surface, such as a vessel bypass or blood occlusion.

The medical devices, support structures, coatings, and covers, described herein, can be biocompatible. As used herein, “biocompatible” means suited for and meeting the purpose and requirements of a medical device, used for either long- or short-term implants or for non-implantable applications. Long-term implants are generally defined as devices implanted for more than about 30 days.

As used herein, “membrane” means a layer of film or multiple layers of film concentrically arranged along a common axis to form a tubular member.

As used herein, “layer” means one or more windings or wraps of film, wrapped in generally the same direction and/or orientation, where the film comprises a single composition. An extruded polymeric material can also be considered a layer.

For example, a stent graft can comprise a graft member comprising a flexible membrane that allows the stent graft to be deployed in a blood vessel and provide a bypass route to avoid vessel damage or abnormalities, such as aneurysms. The membrane of the graft member can comprise one or more layers of material. In accordance with an embodiment, the layers of material are selected to provide a membrane of relatively low thickness, such as, for example, less than 100 microns. In other embodiments, the thickness of the membrane can be in the range of about 20 to about 50 microns, or less.

In accordance with the present disclosure, various characteristics of the membrane of relatively low thickness are comparable to or greater than the membranes of conventional graft members, including, among others, burst strength, abrasion resistance, and maximum load capacity. Stated another way, thinner membranes may be achieved without typically expected trade-offs in other desirable characteristics. For example, the burst strength of a wrapped membrane in accordance with the present disclosure, namely one having a thickness of about 55 microns, can be greater than about 465 kPa, and the maximum load capacity can be, for example, greater than about 60 kilograms.

Other benefits of a graft member comprising a relatively low thickness membrane include increased flexibility and steerability, increased transparency, increased tear resistance, a reduced coefficient of friction, reduced surface tension, and increased crushability, among others.

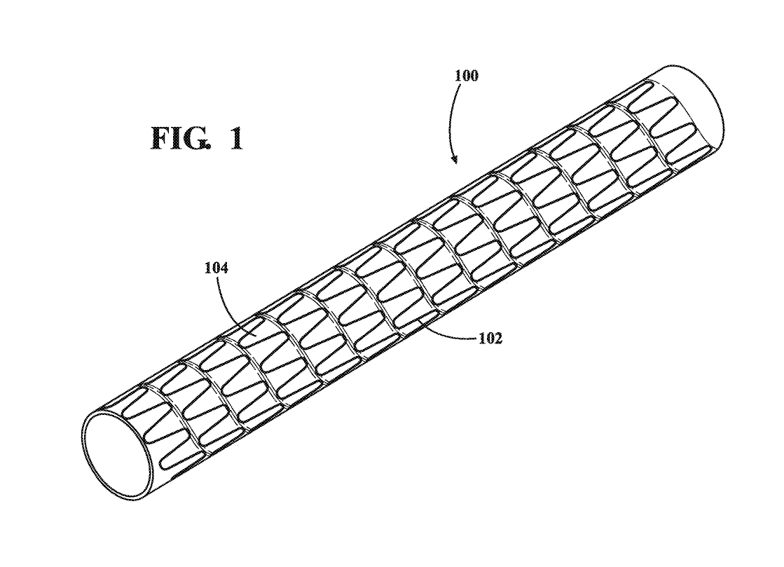

The above being noted, with reference now to FIG. 1, a medical device 100 in accordance with the present disclosure is illustrated. Medical device 100 comprises a stent 102 and a graft member 104. In various embodiments, graft member 104 is affixed to the outside surface of stent 102, such that, once deployed, graft member 104 is in contact with a vessel wall. In other embodiments, graft member 104 is affixed to the inside surface of stent 102, such that, once deployed, graft member 104 is not in contact with the vessel wall. In yet other embodiments, multiple graft members 104 can be utilized, such that one graft member 104 is affixed to the inside of stent 102 and another is affixed to the outside of stent 102.

In various embodiments, stent 102 comprises a biocompatible material. For example, stent 102 can be formed from metallic, polymeric or natural materials and can comprise conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol, and biologically derived materials such as bovine arteries/veins, pericardium and collagen. Stent 102 can also comprise bioresorbable materials such as poly(amino acids), poly(anhydrides), poly(caprolactones), poly(lactic/glycolic acid) polymers, poly(hydroxybutyrates) and poly(orthoesters). Any material which is biocompatible and provides adequate support for medical device 100 is in accordance with the present disclosure.

Stent 102 can comprise, for example, various configurations such as rings, cut tubes, wound wires (or ribbons) or flat patterned sheets rolled into a tubular form. However, any configuration of stent 102 which can be implanted in the vasculature of a patient is in accordance with the present disclosure.

In various embodiments, graft member 104 comprises a biocompatible material that provides a lumen for blood flow within a vasculature. For example, graft member 104 can comprise a composite material having a flexible matrix. In such configurations, the flexible matrix can comprise, for example, expanded polytetrafluoroethylene (ePTFE), pebax, polyester, polyurethane, fluoropolymers, such as perfouorelastomers and the like, polytetrafluoroethylene, silicones, urethanes, ultra high molecular weight polyethylene, aramid fibers, silk, and combinations thereof. Other flexible matrices can include high strength polymer fibers such as ultra high molecular weight polyethylene fibers (e.g., Spectra®, Dyneema Purity®, etc.) or aramid fibers (e.g., Technora®, etc.). Any graft member 104 that provides a sufficient lumen for blood flow within a vasculature is in accordance with the present disclosure.

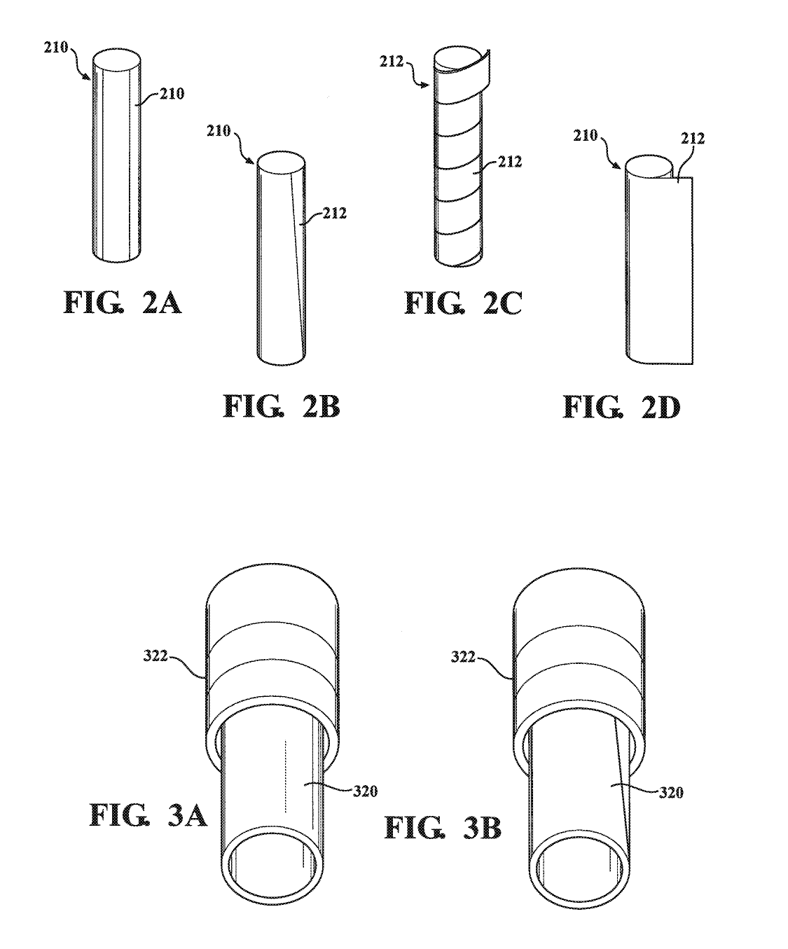

As previously described, a layer comprises one or more windings (or wraps) of film, wherein the film is wrapped in generally the same orientation and comprises the same material. With reference to FIGS. 2A-2D, various methods of preparing a layer of graft member 104 are illustrated. For example, FIG. 2A illustrates a layer of material comprising a flexible matrix, wrapped such that the direction of wrapping is substantially parallel to a central axis of the lumen of graft member 104. FIG. 2B illustrates a layer of material wrapped such that the direction of wrapping is at a relatively low angle (between about 0 and about 30 degrees) above the central axis of the lumen of graft member 104. FIG. 2C illustrates a layer of material wrapped such that the direction of wrapping is at a relatively high angle (between about 30 and about 85 degrees) above the central axis of the lumen of graft member 104. FIG. 2D illustrates a layer of material wrapped such that the direction of the wrapping is substantially perpendicular to the central axis of the lumen of graft member 104.

In various embodiments, the orientation of the wrapping of the material and hence, the longitudinal or machine direction, can be chosen to give one or more different characteristics to the layer. For example, the burst strength of a layer can be improved by increasing the angle of wrapping relative to the central lumen of graft member 104. Further, the maximum load capability of the layer can be improved by reducing the angle of wrapping relative to the central lumen of graft member 104. Other characteristics, such as transmural leakage, abrasion resistance, and adhesion, can be improved by selecting appropriate wrapping orientations that correspond with the desired characteristics.

In various embodiments, graft member 104 can comprise a composite material having a flexible matrix and an elastomeric component. An elastomeric component can comprise, for example, perfluoromethyl vinyl ether (PMVE), such as described in U.S. Pat. No. 7,462,675. Other biocompatible polymers which may be suitable for use in embodiments may include, but are not limited to, the group of urethanes, silicones, copolymers of silicon-urethane, styrene-isobutylene copolymers, polyisobutylene, polyethylene-co-poly(vinyl acetate), polyester copolymers, nylon copolymers, fluorinated hydrocarbon polymers and copolymers or mixtures of each of the foregoing. In such configurations, the flexible matrix is imbibed with the elastomeric component. However, any elastomeric component that is biocompatible and can be imbibed by a suitable flexible matrix is in accordance with the present disclosure.

For example, graft member 104 can comprise a composite material having a flexible matrix of ePTFE imbibed with a TFE/PMVE copolymer, such that the resulting composite material is about 30 wt % of ePTFE and about 70 wt % of TFE/PMVE copolymer. In other embodiments, graft member 104 can comprise a composite material having a flexible matrix of PET imbibed with a TFE/PMVE copolymer, such that the resulting composite material is about 72 wt % of PET and about 28 wt % of TFE/PMVE copolymer. Although discussed in relation to embodiments having specific compositions and weight percentages, the use of any suitable biocompatible composite material, including a combination of a flexible matrix and one or more elastomeric components, is within the scope of the present disclosure.

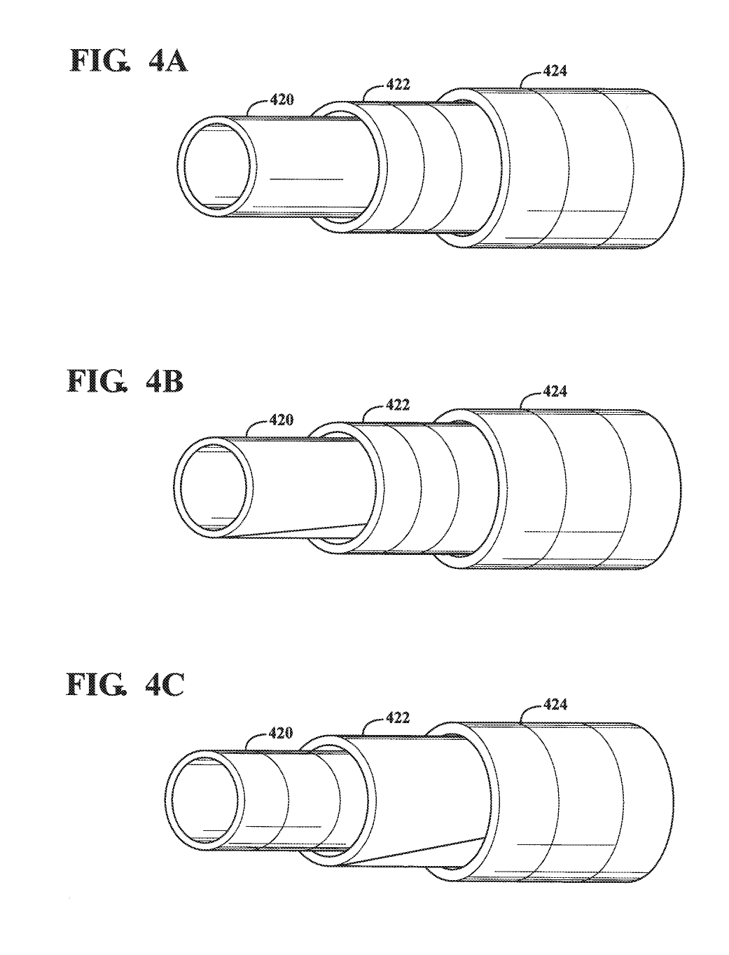

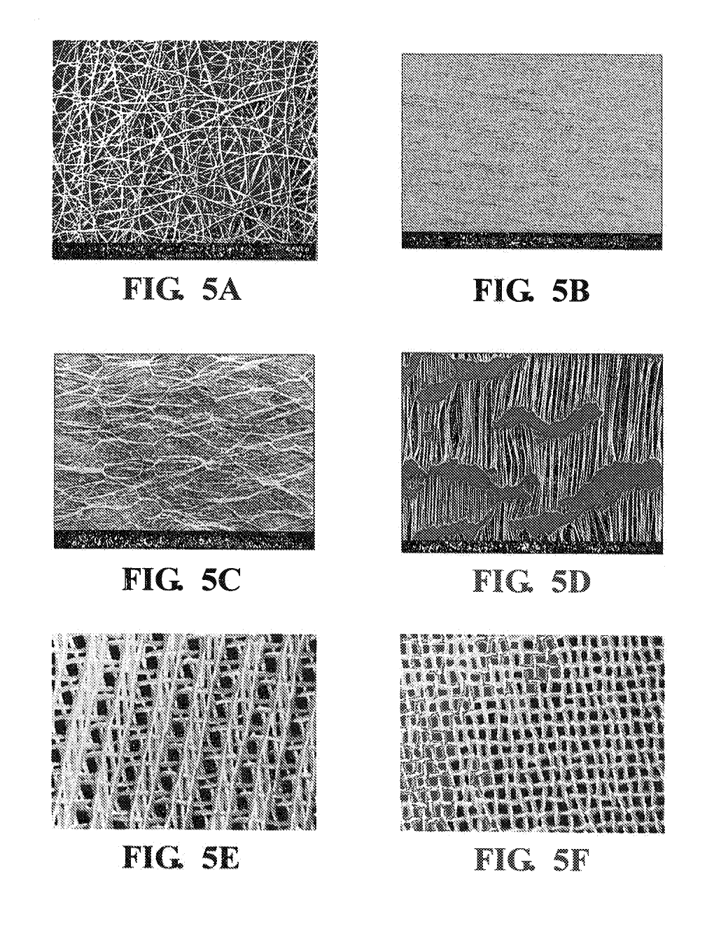

With reference now to FIGS. 3A and 3B, in various embodiments, graft member 104 comprises two layers of material. For example, FIGS. 3A and 3B illustrate a first layer 320 and a second layer 322. In such configurations, second layer 322 concentrically surrounds first layer 320.

As illustrated in FIG. 3A, first layer 320 can comprise an extruded flexible matrix. For example, first layer 320 can comprise extruded ePTFE. As illustrated in FIG. 3B, first layer 320 can comprise a flexible matrix in the form of a wrapped film. As illustrated in FIGS. 2A-2D, the film can be wrapped in any manner that provides a suitable lumen for blood flow and imparts graft member 104 with the desired characteristics, such as burst strength, maximum load, and abrasion resistance, among others.

In various embodiments, second layer 322 can comprise a wrapped flexible matrix. For example, second layer 322 can comprise a material, such as ePTFE, FEP, woven materials such as PET, polyester, nylon, and silk, or any other suitable flexible matrix. In various embodiments, second layer 322 further comprises an elastomeric component, such as perfluoroalkylvinylether.

In various embodiments, second layer 322 is wrapped in one or more windings around an extruded first layer 320. As illustrated in FIG. 3A, second layer 322 can comprise windings that are oriented substantially perpendicularly to a central axis extending longitudinally through first layer 320. In other embodiments, second layer 322 can comprise windings substantially parallel to a central axis extending longitudinally through first layer 320. In yet other embodiments, second layer 322 can comprise windings wrapped at a relatively low angle (between about 0 and about 30 degrees) above the central axis extending longitudinally through first layer 320. Second layer 322 can also comprise windings wrapped at a relatively high angle (between about 30 and about 85 degrees) above the central axis extending longitudinally through first layer 320. However, any angle of wrapping of second layer 322 relative to first layer 320 is in accordance with the present disclosure.

With reference now to FIGS. 4A-4C, in various embodiments, graft member 104 further comprises a third layer of material. For example, FIG. 4A illustrates a first layer 420, a second layer 422, and a third layer 424. In such embodiments, first layer 420 can comprise any suitable flexible matrix, as described in relation to FIGS. 2A-2D, 3A, and 3B. Similarly, second layer 422 can comprise any suitable flexible matrix, as described in relation to FIGS. 3A and 3B.

In various embodiments, third layer 424 can comprise a film of flexible matrix wrapped in one or more windings around first layer 420. As illustrated in FIG. 4A, third layer 424 can comprise windings that are oriented substantially perpendicularly to a central axis extending longitudinally through first layer 420. In other embodiments, third layer 424 can comprise windings substantially parallel to a central axis extending longitudinally through first layer 420. In yet other embodiments, third layer 424 can comprise windings wrapped at a relatively low angle (between about 0 and about 30 degrees) above the central axis extending longitudinally through first layer 420. Third layer 424 can also comprise windings wrapped at a relatively high angle (between about 30 and about 85 degrees) above the central axis extending longitudinally through first layer 420. However, any angle of wrapping of third layer 424 relative to first layer 420 is in accordance with the present disclosure.

FIG. 4A illustrates graft member 104 comprised of a first layer 420, second layer 422, and third layer 424. In the illustrated embodiment, first layer 420 comprises an extruded flexible matrix. Second layer 422 comprises a film wrapped substantially perpendicular to first layer 420. Third layer 424 comprises a film wrapped substantially perpendicular to first layer 420.

FIG. 4B illustrates a graft member 104 comprised of a first layer 420, second layer 422, and third layer 424. In the illustrated embodiment, first layer 420 comprises a film wrapped at a relatively low level relative to a central axis of the lumen of first layer 420. Second layer 422 comprises a film wrapped substantially perpendicular to first layer 420. Third layer 424 comprises a film wrapped substantially perpendicular to first layer 420.

FIG. 4C illustrates a graft member 104 comprised of a first layer 420, second layer 422, and third layer 424. In the illustrated embodiment, first layer 420 comprises a film wrapped substantially perpendicular relative to a central axis of the lumen of first layer 420. Second layer 422 comprises a film wrapped at a relatively low level relative to a central axis of first layer 420. Third layer 424 comprises a film wrapped substantially perpendicular to first layer 420. However, third layer 424 can comprise any material, such as an extruded flexible matrix or a film of flexible matrix with or without an elastomeric component, suitable for providing sufficient strength and support to graft member 104.

It should be noted that although described in double and triple layer embodiments, graft member 104 can comprise any number of layers of flexible matrices, with or without elastomeric components, suitable for providing sufficient strength and support for blood flow through the lumen of graft member 104.

In accordance with the present disclosure, the use of an elastomeric component combined with a flexible matrix allows for a broader selection of materials for use in forming the various layers of graft member 104. As discussed in relation to the various film wrapping orientations, the materials selected for the flexible matrices and elastomeric components of any of the layers described above can be selected to impart particular properties to graft member 104.

With reference now to FIGS. 5A-5F, scanning electron microscope (SEM) images of various materials suitable for first layers 320 and 420, second layers 322 and 422, and/or third layer 424 are illustrated. FIG. 5A illustrates a polymeric material comprising a biaxially oriented flexible matrix of porous ePTFE generally described in U.S. Pat. No. 7,306,729. FIG. 5B illustrates a relatively high-density and low-permeability ePTFE flexible material with thermoplastic FEP on the opposing surface (not shown). FIG. 5C illustrates a predominately uniaxially oriented polymeric material comprising a flexible matrix of ePTFE. FIG. 5D illustrates a polymeric material comprising a flexible matrix of ePTFE that was extruded in tubular form and is uniaxially oriented. FIG. 5E illustrates a woven polyester fabric with an average pore size of 200 microns. FIG. 5F illustrates a woven polyester fabric with an average pore size of 100 microns.

In various embodiments, layers of flexible matrix, with or without elastomeric components, can be selected to impart graft member 104 with, in addition to being relatively thin, one or more additional desired characteristics. For example, one or more layers can comprise material selected to provide sufficient burst strength to graft member 104. Other desirable characteristics of graft member can include tensile strength, stretch, density, low permeability of fluids, transparency, and maximum load, among others.

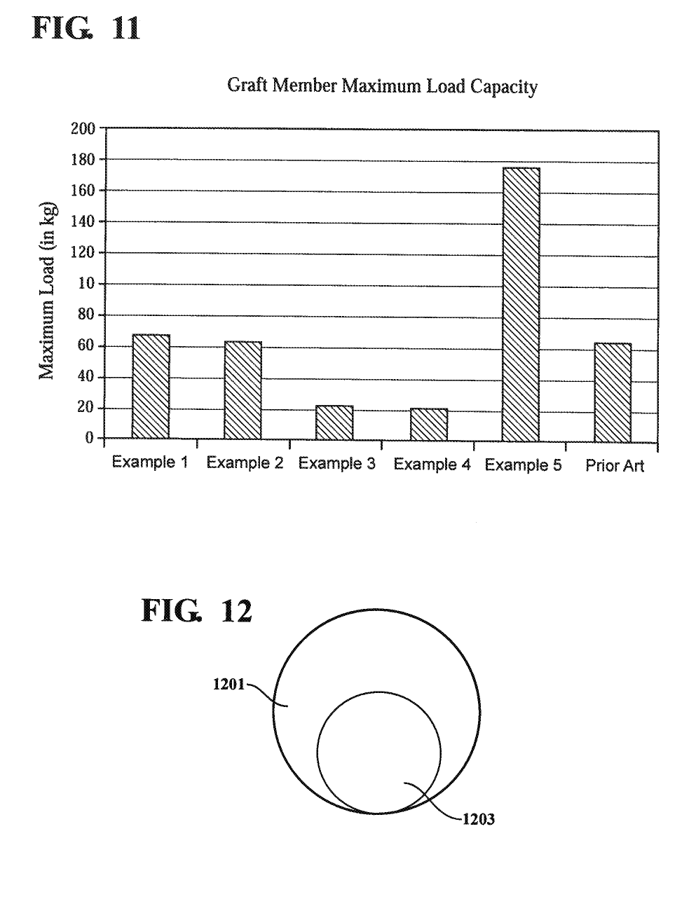

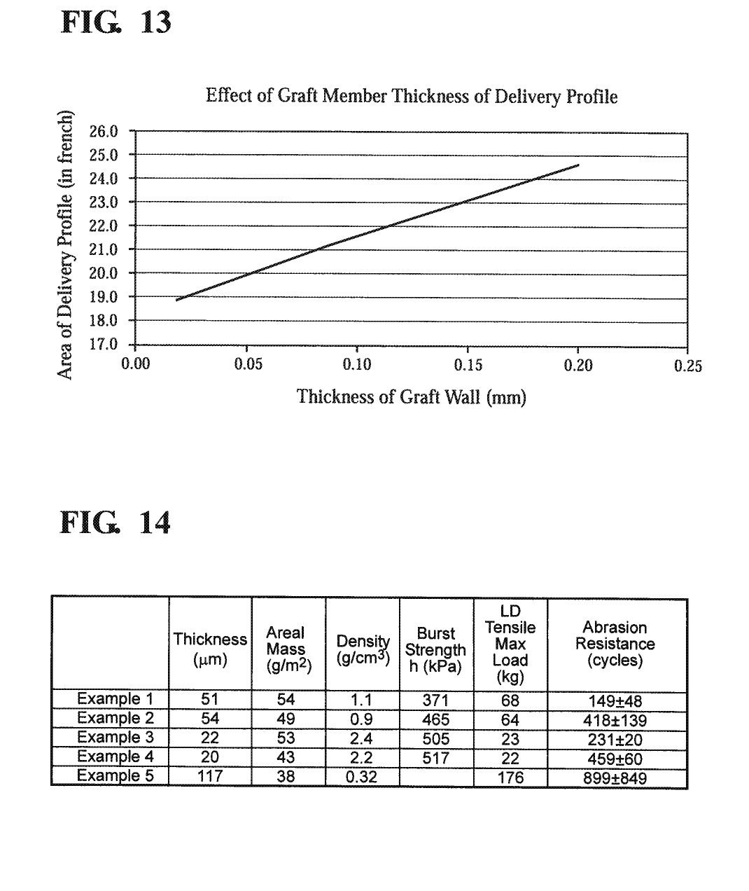

As previously discussed, as the thickness of graft member 104 is decreased, the cross sectional delivery profile area of corresponding medical device 100 is also reduced. With reference now to FIG. 13, the relationship between the thickness of graft member 104 and cross sectional delivery profile area of medical device 100 is illustrated. In regards to a particular embodiment, and with reference now to FIG. 12, the cross sectional delivery profile area of medical device 100 in accordance with the present disclosure is compared to the cross sectional area of a conventional stent graft. For example, prior art cross sectional area 1201 corresponds to a cross sectional delivery profile area of a prior art stent graft having a graft member with a thickness of approximately 120 microns. Relatively low thickness graft member cross sectional delivery profile area 1203 corresponds to the cross sectional delivery profile area of a stent graft having a graft member with a thickness of approximately 25 microns. Thus, the reduction of the thickness of a graft member from 120 microns to 25 microns results in a reduction of cross sectional delivery profile area of the stent graft of approximately 25% or more.

In accordance with the present disclosure, in various embodiments, a medical device can comprise coatings. In various embodiments, the coatings comprise bio-active agents. Bio-active agents can be coated onto a portion or the entirety of the stent and/or graft member for controlled release of the agents once the device is implanted. The bio-active agents can include, but are not limited to, vasodilator, anti-coagulants, such as, for example, warfarin and heparin. Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

In various embodiments, a medical device can be deployed using any suitable device delivery system. The device delivery system can comprise one or more catheters, guidewires, or other suitable conduits for delivering an elongated segment to a treatment region. In these embodiments, the catheters, guidewires, or conduits can comprise lumens configured to receive inputs and/or materials from the proximal end of the medical device delivery system and conduct the inputs and/or materials to the elongated segment at the treatment region.

In various embodiments, various components of the devices disclosed herein are steerable. For example, during deployment at a treatment site, one or more of the elongated segments can be configured with a removable steering system that allows an end of the elongated segment to be biased or directed by a user. A removable steering system in accordance with various embodiments can facilitate independent positioning of an elongated segment and can provide for the ability of a user to accomplish any of the types of movements previously described, such as longitudinal movement, rotational movement, lateral movement, or angular movement.

### EXAMPLES

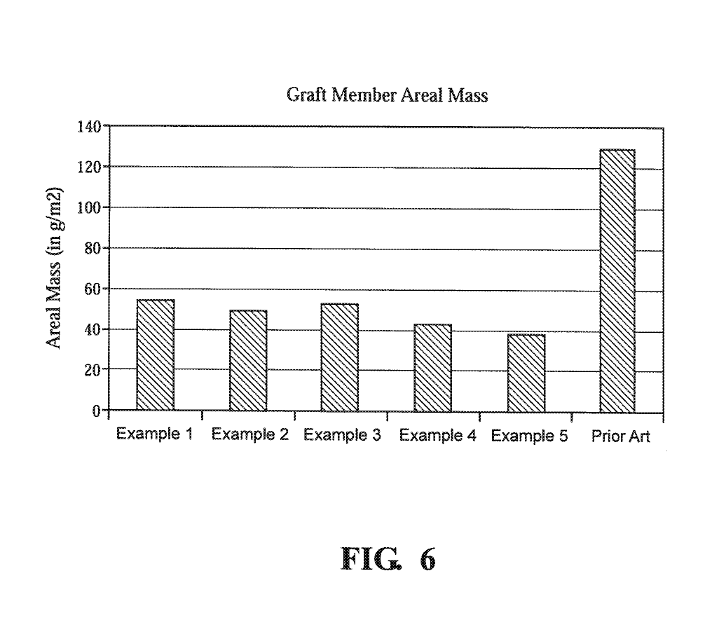

Examples 1-5 consist of graft members constructed in accordance with various embodiments of the present disclosure. Each example graft member was subjected to a number of tests to compare the attributes of each of the graft members, as well as to the membrane of a prior art stent graft. The results of these tests are illustrated in FIGS. 6-11.

Example 1 comprises a first layer of an ePTFE tube pulled onto a 32.3 mm round stainless steel mandrel. Three windings of dense ePTFE/FEP film were applied with the FEP side oriented toward the ePTFE tube, the windings oriented circumferentially to the central axis of the first layer. Next, one and a half windings of 5 cm wide by 0.7 mm thick sacrificial ePTFE tape were applied for compression. The sample was heated in an ESPEC Super-Temp STPH-201 oven (Tabai Espec Corp., Osaka, Japan) set to 320° C. for approximately 30 minutes. After cooling to room temperature, the sacrificial material and mandrel were removed from the tube construct. This configuration is generally illustrated in FIG. 3A. The resulting membrane is about 51 microns thick.

Example 2 comprises a first layer of an ePTFE tube pulled onto a 32.3 mm round stainless steel mandrel. Twenty wraps of the ePTFE/elastomer film were applied to the ePTFE tube, the windings oriented circumferentially to the central axis of the first layer. The ePTFE component constitutes about 30 wt % of the ePTFE/elastomer film, and has a microstructure consistent with that shown in FIG. 5A. The elastomer component constitutes about 70 wt % of the ePTFE/elastomer film, and comprises a TFE/PMVE copolymer that consists essentially of between about 35 and 30 wt % TFE and complementally about 65 and 70 wt % PMVE. Next, one and a half windings of 5 cm wide by 0.7 mm thick sacrificial ePTFE tape were applied for compression. The sample was heated in an ESPEC Super-Temp STPH-201 oven (Tabai Espec Corp., Osaka, Japan) set to 320° C. for approximately 30 minutes. After cooling to room temperature, the sacrificial material and mandrel was removed from the tube construct. This configuration is generally illustrated in FIG. 3A. The resulting membrane is about 54 microns thick.

Example 3 comprises a first layer of one winding of an ePTFE/FEP film applied to a 32.3 mm stainless steel mandrel with the FEP side oriented away from the mandrel. Three windings of dense ePTFE/FEP film were applied with the FEP side oriented toward the ePTFE tube, the windings oriented circumferentially to the central axis of the first layer. One and a half windings of 5 cm wide by 0.7 mm thick sacrificial ePTFE tape were applied for compression. The sample was then heated in an ESPEC Super-Temp STPH-201 oven (Tabai Espec Corp., Osaka, Japan) set to 320° C. for approximately 30 minutes. After cooling to room temperature, the sacrificial material and mandrel were removed from the tube construct. This configuration is generally illustrated in FIGS. 2D and 3A. The resulting membrane is about 22 microns thick.

Example 4 comprises a first layer of one winding of the ePTFE/FEP film applied to a 32.3 mm stainless steel mandrel with the FEP side oriented away from the mandrel. Twenty wraps of an ePTFE/elastomer film were are applied to the ePTFE tube with the longitudinal direction of the film oriented circumferentially. The ePTFE component of the ePTFE/elastomer film constitutes about 30 wt % of the ePTFE/elastomer film, and has a microstructure consistent with that shown in FIG. 5A. The elastomer component of the film constitutes about 70 wt % of the ePTFE/elastomer film, and comprises a TFE/PMVE copolymer that consists essentially of between about 35 and 30 wt % TFE and complementally about 65 and 70 wt % PMVE. One and a half wraps of 5 cm wide by 0.7 mm thick sacrificial ePTFE tape were applied for compression. The sample was heated in an ESPEC Super-Temp STPH-201 oven (Tabai Espec Corp., Osaka, Japan) set to 320° C. for approximately 30 minutes. After cooling to room temperature, the sacrificial material and mandrel were removed from the tube construct. This configuration is generally illustrated in FIGS. 2D and 3A. The resulting membrane is about 20 microns thick.

Example 5 comprises a plain weave of woven PET material mounted in a 25 cm diameter plastic embroidery hoop to produce a wrinkle-free surface. A brush was used to coat the fabric with a mixture containing about 3 wt % TFE/PVME fluorinated elastomer, such as described in U.S. Pat. No. 7,462,675, and 97 wt % Fluorinert® solvent (a perfluorinated solvent commercially available from 3M, Inc., St. Paul, Minn.). The sample was dried at room temperature and atmospheric pressure for at least 24 hours. The PET component constitutes about 72 wt % of the resulting PET/elastomer film, and the elastomer component constitutes the remaining about 28 wt %. The elastomer is a TFE/PMVE copolymer that consists essentially of between about 35 and 30 wt % TFE and complementally about 65 and 70 wt % PMVE. The resulting PET/elastomer film can be used as a wrapped layer of a graft member. The resulting membrane is between about 113 and about 117 microns thick.

A chart is provided in FIG. 14 summarizing the various properties of Examples 1-5 described above.

With reference to FIG. 6, the areal mass of the graft members of examples 1-4, as well as the membrane of a prior art device, are illustrated. Areal mass for films is measured by weighing a 15 cm by 15 cm swatch using a Mettler Toledo Scale Model AB104, or comparable apparatus. Areal mass for tubes is measured by weighing a 23 cm length of tube with a known diameter using a Mettler Toledo Scale Model AB104, or comparable apparatus. The areal mass is calculated using the following equation:

Areal Mass=(mass of sample/area of sample).

The areal masses of the four example graft membranes are between about 35% and about 45% of the areal mass of the prior art device, but as is shown in Table 1, the graft membranes are notably thinner.

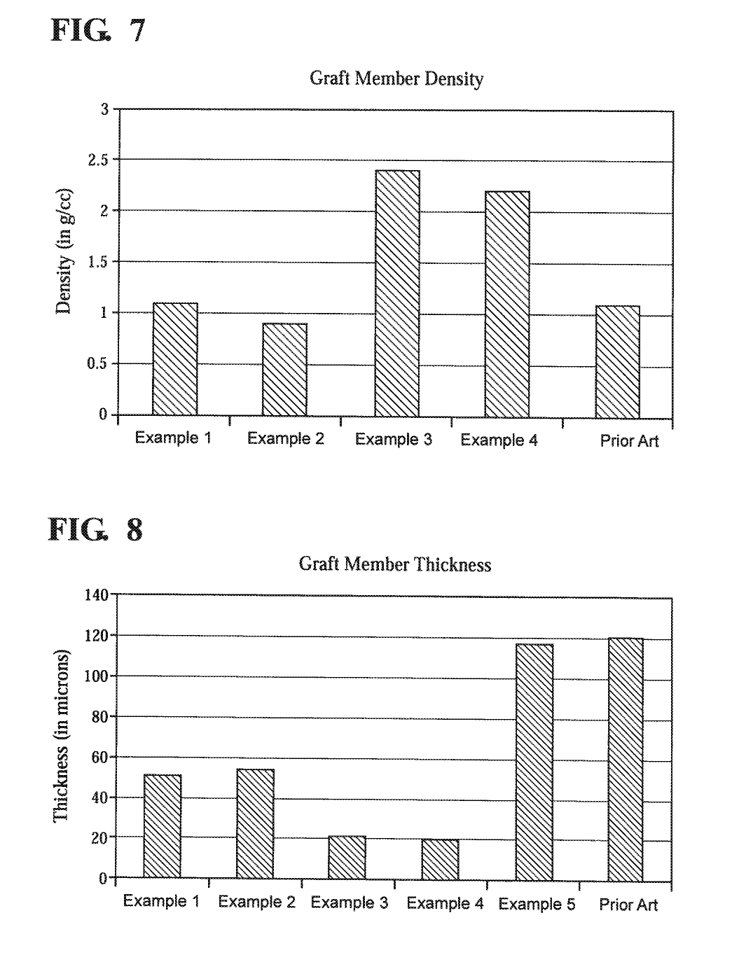

With reference to FIG. 7, the density of the graft members of examples 1-4, as well as the membrane of a prior art device, are illustrated. Despite the relatively low thickness of the example graft membranes, the densities of the example graft membranes are about 90% to about 200% of the density of the prior art device.

With reference to FIG. 8, the thickness of the graft members of examples 1-4, as well as the membrane of a prior art device, are illustrated. The thickness of each graft member was measured using a Mitutoyo snap gauge, code No. 7004 (Mitutoyo Mexicana S.A. de C.V.). However, the thickness can be measured by any suitable gauge or acceptable measurement technique. The thickness of the example graft members ranges from about 20% to about 55% of the thickness of the prior art device.

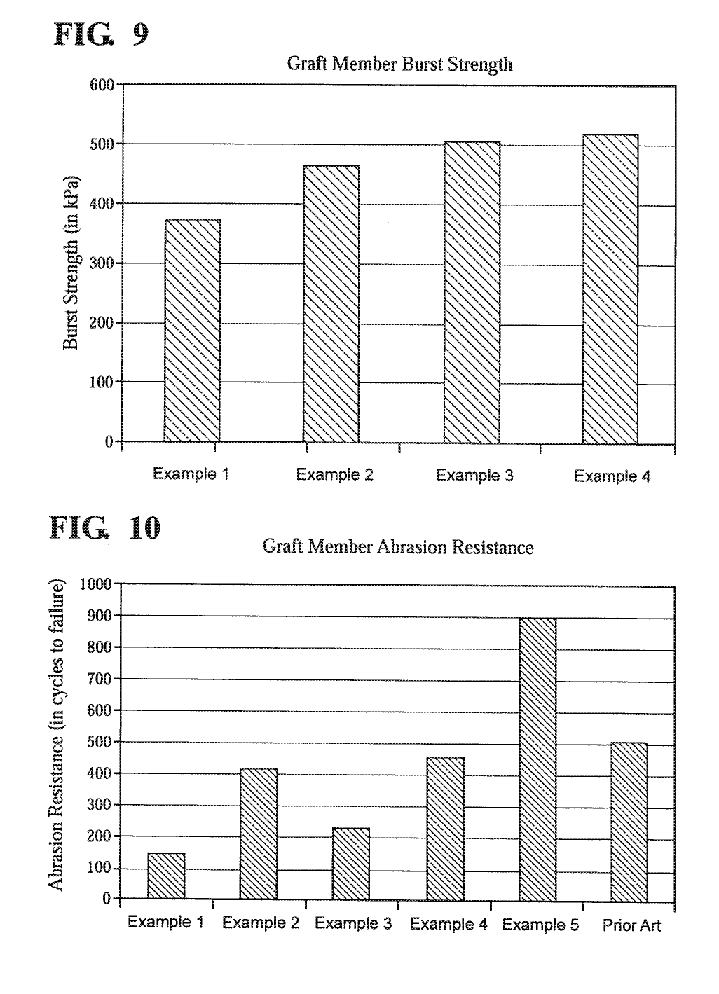

With reference to FIG. 9, the tube burst strength of examples 1-4 are illustrated. To measure the burst pressure or strength of each graft member, the pressure of water required to mechanically rupture a tube is measured. For example, 32.3 mm graft member samples are prepared by lining each sample with a 25.4 mm outer diameter by 0.8 mm thick latex tube. The lined graft members are cut to approximately 10 cm in length. A small metal hose is inserted into one end of the lined graft member and held in place with a clamp to create a water-tight seal. A similar clamp is placed on the other end of the member. Room temperature water is pumped into the graft member to increase the internal pressure at a rate of 69 kPa/s through the metal hose that is connected to an automated sensor that records the maximum pressure achieved before mechanical rupture of the tube sample. Despite the relatively low thickness of the example graft members, burst strengths of the example graft members did not drop proportionately. The high burst strengths of the example graft members 2 and 4 illustrate that despite having thicknesses that are 17% and 46% of the prior art, the example graft members have burst strengths that are 56% and 62% of the prior art, respectively. It should be readily appreciated that burst strengths can be described in terms of hoop or wall stress, where:

burst wall stress=(burst pressure×inside radius)/wall thickness.

With reference to FIG. 10, the relative wire abrasion of examples 1-4, as well as the membrane of a prior art device, are illustrated. To measure the wire abrasion of each graft member, a Repeated Scrape Abrasion Tester (cat. 158L238G1, Wellman Thermal Systems Corp., Shelbyville, Ind.), or comparable apparatus, is used. A 1 cm×5 cm test sample is cut from the graft member, with the 5 cm dimension oriented along the axis of the test sample. The test sample is mounted onto a 3 mm diameter mounting mandrel and held in place by two set-screw type collars on either end. The abrading mandrel used to conduct the test is a 0.44 mm diameter NiTi alloy. A total weight of approximately 280 g is applied to the abrading mandrel as it cycles with an 8.5 mm stroke at a rate of 1 stroke per second. The total number of cycles required for the abrading mandrel to abrade through the sample and contact the mounting mandrel is recorded. The average of at least five measurements is used to determine the final experimental value for the wire abrasion test. Despite the relatively low thickness of the example graft members, the abrasion resistances of the example graft members ranges from about 30% to 100% of the abrasion resistance of the prior art device. The relatively high abrasion resistances of the example graft members illustrates that despite the reduced thickness, the example graft members have a comparable abrasion resistance to the prior art device.

With reference to FIG. 11, the maximum load capacity of examples 1-4, as well as the membrane of a prior art device, are illustrated. The maximum load capacity for each graft member is measured using an INSTRON 4501 tensile test machine equipped with flat-faced grips and a 100 kg load cell, or any comparable tensile testing apparatus. The gauge length is 5.1 cm and the cross-head speed is 10 cm/min. Test samples of 13 cm in length and 2.5 cm in width are created from each graft member. Each test sample is weighed using a Mettler Toledo Scale Model AB104, or a comparable apparatus. The thickness of the test samples is measured using the Mitutoyo snap gauge, or a comparable apparatus. The samples are then tested individually with the INSTRON 4501 tensile tester. Despite the relatively low thickness of the example graft members, the maximum load capacities of the example graft members range from about 30%% to about 105% of the burst strength of the prior art device. The high maximum load capacities of the example graft members illustrates that despite the reduced thickness, the example graft members have a comparable maximum load capacity to the prior art.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications can be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

1.-13. (canceled)

14. An endoluminally deliverable implantable device, comprising:
a biocompatible tubular member formed from a composite having a first layer comprising a first flexible matrix; and, a second layer comprising: an elastomeric component and a second flexible matrix, wherein the second layer surrounds at least a portion of the first layer, and
wherein the areal density of the biocompatible tubular member is less than 100 g/m2, and
wherein the elastomeric component of the second flexible matrix accounts for 10-70% of the total mass of the biocompatible tubular member.

15. The endoluminally deliverable implantable device of claim 14, wherein the first flexible matrix comprises at least one wrapped membrane.

16. The endoluminally deliverable implantable device of claim 15, wherein the at least one wrapped membrane of the first flexible matrix comprises one of ePTFE, ePTFE co-polymer, polyester, nylons, and FEP.

17. The endoluminally deliverable implantable device of claim 14, wherein the first flexible matrix comprises an extruded polymeric material.

18. The endoluminally deliverable implantable device of claim 17, wherein the extruded polymeric material comprises at least one of ePTFE, ePTFE co-polymer, and FEP.

19. The endoluminally deliverable implantable device of claim 14, wherein the second flexible matrix comprises at least one wrapped membrane.

20. The endoluminally deliverable implantable device of claim 19, wherein the at least one wrapped membrane comprises one of an ePTFE and FEP laminate.

21. The endoluminally deliverable implantable device of claim 14, wherein the elastomeric component of the second layer is TFE/PMVE copolymer.

22. The endoluminally deliverable implantable device of claim 14, wherein first layer consists of the first flexible matrix without an elastomeric component.

23. The endoluminally deliverable implantable device of claim 14, wherein the first layer defines a lumen of the tubular member.

24. The endoluminally deliverable implantable device of claim 14 including a stent.

25. The endoluminally deliverable implantable device of claim 24, wherein the stent is sandwiched between the first layer and the second layer.

26. The endoluminally deliverable implantable device of claim 14 including a third layer comprising a third flexible matrix.

27. The endoluminally deliverable implantable device of claim 26, wherein the third layer surrounds at least a portion of the second layer.

28. The endoluminally deliverable implantable device of claim 26, wherein the third flexible matrix comprises one of ePTFE, ePTFE co-polymer, and FEP.

29. A method for manufacturing an implantable device for guiding blood flow, said method comprising:
forming a biocompatible tubular member by creating a first layer comprising a first flexible matrix; and creating a second layer comprising an elastomeric component and second flexible matrix, wherein the second layer surrounds at least a portion of the first layer, wherein the areal density of the biocompatible tubular member is less than 100 g/m2, and the elastomeric component of the second layer accounts for 10-70% of the total mass of the biocompatible tubular member.

30. The method of claim 29, further comprising a step of surrounding the second layer with a third layer.

31. The method of claim 29, wherein the first flexible matrix comprises at least one wrapped membrane.

32. The method of claim 31, wherein the at least one wrapped membrane of the first flexible matrix comprises one of ePTFE, ePTFE co-polymer, polyester, nylons, and FEP.

33. The method of claim 29, wherein the first flexible matrix comprises an extruded polymeric material.

34. The method of claim 33, wherein the extruded polymeric material comprises at least one of ePTFE, ePTFE co-polymer, and FEP.

35. The method of claim 29, wherein the second flexible matrix comprises at least one wrapped membrane.

36. The method of claim 35, wherein the at least one wrapped membrane comprises an FEP laminate.

37. The method of claim 31, wherein first layer consists of the first flexible matrix without an elastomeric component.

38. The method of claim 29, wherein the elastomeric component of the second layer is TFE/PMVE copolymer.

39. The method of claim 29, further comprising a step of affixing the implantable device to a stent.
